(19) United States
(12) Patent Application Publication
(10) Pub. No.: US 2023/0075885 A1
(43) Pub. Date: Mar. 9, 2023

(54) COMPOSITIONS AND APPLICATIONS THEREOF

(71) Applicant: Nasaleze Patents Limited, Douglas (GB)

(72) Inventors: Todor A. Popov, Sofia (BG); Peter David Josling, Hailsham (GB)

(73) Assignee: Nasaleze Patents Limited, Douglas, GB (GB)

(21) Appl. No.: 17/760,390

(22) PCT Filed: Feb. 11, 2021
(86) PCT No.: PCT/GB2021/000013

§ 371 Date: Aug. 9, 2022

(30) Foreign Application Priority Data
Feb. 12, 2020 (GB) GB2001906.3
Aug. 27, 2020 (GB) GB2013432.6

(51) Int. Cl. A61K 9/16; A61K 47/46; A61K 31/675; A61K 31/352; A61K 41/00; A61K 38/21; A61K 31/215; A61K 31/7056; A61K 31/513; A61K 36/16; A61K 31/07; A61K 31/375; A61K 31/355; A61K 36/258; A61K 36/82; A61K 31/522; A61K 31/122; A61K 36/23

(52) U.S. Cl. CPC A61K 9/1652; A61K 47/46; A61K 31/675; A61K 31/352; A61K 41/0004; A61K 38/212; A61K 38/215; A61K 31/215; A61K 31/7056; A61K 31/513; A61K 36/16; A61K 31/07; A61K 31/375; A61K 31/355; A61K 36/258; A61K 36/82; A61K 31/522; A61K 31/122; A61K 36/23; A61K 9/1694

(57) ABSTRACT

Compositions in homogenised powder form consisting of hydroxypropyl methylcellulose particles, and at least one chemical signalling agent in particle form, and optionally a biologically active agent, wherein the homogenised powder comprises particles having a mean particle diameter of ≥20 μm to ≤500 μmm uses and kits therefor.

The present invention relates to dry powder compositions for administration to the nasal tract and uses therefor. In particular, the compositions of the invention comprise powdered hydroxypropyl methylcellulose (pHPMC) particles of a defined mean particle size, a signalling agent, and optionally, a biologically active agent.

Allergic rhinitis (AR) is a global health problem which affects up to 25% of the adult population in industrialised countries and more than 40% of children and is thought to be responsible for economic losses amounting to some $2 to $5 billion per annum in the USA alone¹.

It is known from, for example, U.S. Pat. No. 8,202,550 that HPMC powders, and the gel that forms when they are administered to the nasal tract, represent an effective means of intranasally administering therapeutic agents, and in particular, herbal, or homeopathic agents. This prior art invention teaches that a therapeutic agent included in a composition as described therein does produce a therapeutic effect. Upon contact with the nasal mucosa when pHPMC is administered to the nasal cavity, a protective gel is formed which slowly releases moisture and any therapeutic agent which is co-administered. This results in a lasting therapeutic effect, with beneficial effects of the therapeutic agent reported for over 6 hours following administration². In comparison, administration of active agents to the nasal tract in conventional intranasal compositions (such as liquid compositions or powder compositions comprising little or no cellulose) typically provides therapeutic effects of relatively short (below 4 hours) duration. However, there is no disclosure of mean particle size in U.S. Pat. No. 8,202,550.

The prior art further teaches or alludes to the use of HPMC powdered compositions for treating allergic rhinitis (AR) and other respiratory diseases, including certain viral diseases but with no data about coronavirus disease, and in particular, respiratory disease associated with SARS-CoV-2 virus. However, the prior art makes no reference to the mean particle size of powdered HPMC prior to its formation into a gel.

Diethart B. et al Nat. Sci. 2010; Vol. 2 No.2:79-84 teaches the diffusion of a house dust mite allergen through HPMC and agar gels, and thus the potential of using HPMC to block uptake of the allergen³. There is no reference to the mean particle size of powdered HPMC prior to its formation into a gel.

A study in Russia by M. K. Erofeeva et al (https://medi.ru/info/7023/) points to HPMC being useful in preventing influenza in children⁴. However, there was no indication of the mean particle size used in the dry powder HPMC containing formulations used in the study.

Defective nasal barrier function is implicated in allergic rhinitis which results in persistent inflammation and clinical symptoms, among which congestion plays a prominent role. In a recent study by Valerieva, A. et al Allergy Asthma Proc 36:1-6, 2015; doi: 10.2500/aap.2015.36.3879, it was shown that administering HPMC after administration of oxymetazoline provided relief for a sample of patients who were known to be sensitive to at least one of a panel of perennial allergens². Owing to the statistical method used, short duration of the study and small sample size the authors stated that further research was needed. There is no apparent reference to mean particle size in this study and it does not appear that a signalling agent was used.

An article by Popov T. A. et al on HPMC powder for the prevention and management of nasal symptoms (Popov T. A. et al Expert Review of Respiratory Medicine, 2017 Vol. 11. No. 11. 885-892 (https://doi.org/10.1080/17476348.2017.1375408) reported that HPMC provided a natural barrier to pollen allergens and noxious agents⁵. The article itself and a number of studies cited therein involved various plant pollens and house dust mite allergens but did not refer to the mean particle size of the powdered formulations employed.

HPMC powder per se and HPMC powder containing peppermint and wild garlic has been shown to decrease viral titre of H5N1 in SPEV cell culture in an in vitro study when compared to controls (Lvov DK and Deryabin PG Virucidal Activity of Nasaleze (Nasaval) and Nasaleze Travel (Nasaleze Plus) in Cell Cultures Infected with Pathogenic Avian Flu virus (H5N1), 2010, European Journal for Nutraceutical Research 1-8, www.phytomedcentral.org)⁶. There is no reference to mean particle size and what effect size may have on the efficacy of the HPMC powder as a blocking agent to viral uptake.

HPMC powder and the addition of stabilised allicin extract from garlic was shown in an evaluation of a particular strain of methicillin resistant Staphylococcus aureus, namely UEL301, to exhibit significant biological activity at various formulations (Cutler University of East London March 2003). There is no reference to mean particle size and what effect size may have on the efficacy of the HPMC powder as a blocking agent to bacterial removal.

Powdered compositions of HPMC including only signalling agents and/or biologically active agents having a mean particle size as described herein and that are capable of forming gels in contact with moisture have been found to be more efficient at controlling or containing viral infection (size range of virus particles from 0.005 μm to 0.3 μm); allergic rhinitis (AR) brought on by airborne particles such as pollen grains (size range from 10 μm to 1000 μm); allergic reactions caused by inhalation of particles from biological sources such as house dust mites (size range from 100μm to 300μm); and allergic reactions due to inhalation of airborne pollution particles (size range from 1 μm to 150 μm) for example, PM_{2.5} to PM₁₀, than compositions of the prior art⁷. This is surprising given the wide range in size of particles that are inhaled and the range in mean particle size of HPMC powders of the invention.

In view of the rise of two recent outbreaks of deadly coronavirus disease in the Middle East and a third in the Far East where a new coronavirus type that infects humans has now evolved, there is a pressing need to provide formulations that are capable of at least slowing down, blocking and/or negating the spread of such viruses before they can take hold in the host organism. An additional problem with the new coronavirus strain (2019-n CoV, aka SARS-COV-2) from the Far East is that it can be present in the host (humans) for about two weeks before symptoms appear. Should the compositions of the present invention be applied prophylactically and/or curatively to the nasal lining through regular insufflation, containment of the disease may be possible.

The advantages alluded to above and other advantages will become apparent from the following description.

Previously, it was thought that a mean particle size should range from ≥5 μm to ≤500 μm. However, new data indicates that optimum mean particle size lies in the range ≥20 μm to ≤500 μm as detailed herein.

It has now been found that a mean particle size of dry powder compositions of the invention lying within the range from about 20 μm to about 500 μm, preferably within the range ≥60 μm to ≤150 μm, more preferably from 80 to 125 μm is surprisingly efficient at trapping aerial born allergens in a gel. Furthermore, the dry powder compositions of the invention designed for nasal application show promise for the delivery of drugs against aerial-borne viruses, such as coronaviruses and others.

According to the present invention, there is provided a composition in the form of a dry homogenised powder consisting of two or more components selected from
i) hydroxypropyl methylcellulose particles; and
ii) at least one chemical agent selected from signalling agents; and/or
iii) one or more biologically active agents,
   wherein the homogenised dry powder particles have a mean particle size of ≥20 μm to ≤500 μm.

Powdered compositions of the invention preferably have a mean particle size in the range of 20 to 500 μm, preferably from 60 to 150 μm, more preferably from 80 to 125 μm, such as 86 μm +/− 15 μm, depending on added signalling agent and/or added biologically active agent.

The compositions of the invention are designed for application to the nasal mucosa through insufflation via the nose.

Compositions of the invention must be able to form gels on contact with moisture, as illustrated in the accompanying examples. The compositions of the invention should not contain additives that may or could substantially interfere with their ability to form gels on contact with moisture, such as additives that can significantly lower the pH of the nasal mucosa. On contact with the nasal mucosa, the dry powder particles of the invention absorb moisture and thereby form a gel matrix on the surface thereof. The function of the gel is considered to be at least twofold: firstly, it acts as a physical barrier to the uptake of small particulates such as aerial borne allergens and viruses through the nasal mucosa and secondly it permits the diffusion of drug or drugs of choice across the nasal mucosal cells and into the bloodstream. It is thought that during the hydration of dry powdered compositions of the invention a gel matrix is formed through contact with moisture in which larger particles and smaller particles combine to form a molecular net or molecular matrix wherein the smaller particles occupy spaces or gaps between larger particles and so contribute to gel formation, helping the larger particles to subsume together more easily. Particulate matter becomes trapped in the gel and is largely unable to pass over the mucosal membrane.

Compositions of the invention can include a biologically active agent selected from pharmaceutical, herbal, and homeopathic agents. Suitable homeopathic and herbal agents may be selected from St John's Wort, valerian extract, ginkgo biloba extract, vitamins A, E or C, garlic, lime, one or more pro-biotics, ginger, ellagic acid, echinacea, Swedish flower pollen, black walnut hulls, lemongrass, wormwood, grapefruit seed extract, broccoli, digestive enzymes, hyaluronic acid, astragalus, rosehips, gentian, hypericum, horse chestnut, ginseng, green tea, phosphatidyl serine, phosphatidyl choline, citrus, pycnogenol, caffeine, quercitin, co-enzyme Q10, yarrow, tea tree, noni juice, lipase, fructo-oligosaccharide, inulin, black cumin, stabilised allicin, or any combination thereof.

Compositions of the invention can include a biologically active agent selected from the pharmaceutical antiviral agents: Type I (α, β) interferons (IFN), such as IFN-β, IFN-β-1b, Type II (y) and Type III (A) interferons, favipiravir,(aka favilovir, T-705, and Avigan) available from Fujifilm Toyama Chemical, remdesivir, ozeltamivir, zanamivir, ribavirin, lopinavir, combination of lopinavir-ritonavir and IFNβ-b, monoclonal and (camel) polyclonal neutralising antibodies and macrolides, such as ivermectin, plant alkaloids such as colchicine, and the like. A compound that shows potential for use against coronaviruses in general is K22, structural name (Z)-N-(3-(4-(4-bromophenyl)-4-hydroxypiperidin-1-yl)-3-oxo-1-phenylprop-1-en-2-yl) benzamide, available from ChemDiv (San Diego, Calif.: catalog number 4295-0370). This compound targets membrane-bound viral RNA synthesis and shows potent inhibition in diverse coronaviruses, including MERS virus. Further suitable biologically active agents include isolated griffithsin lectin proteins of about 121 amino acids extractable from a red algae seaweed such as Griffithsia, and biologically active, isolated antiviral analogues thereof, and natural seaweed extracts containing it (Journal of Virology 2010, O'Keefe, B. R et al ‘Broad Spectrum In Vitro Activity and In Vivo Efficacy of the Antiviral Protein Griffithsin against Emerging Viruses of the Family Coronaviridae’⁸. Published online DOI: 10.1128/JVI.02322-09; Marine Drugs 2019 October; 17(10): 567, Choongho Lee ‘Griffithsin a highly potent Broad Spectrum Anti-Viral Lectin from Red Algae: From Discovery to Clinical Application’ Published online 2019 Oct. 6. doi: 10.3390/md17100567.

Compositions according to the invention may contain a pHPMC, a biologically active agent as herein defined and a signalling agent or additive such as menthol, strawberry, mint, spearmint, peppermint, eucalyptus, lavender, and citrus, or any combination thereof. Examples of citrus may include lemon, lime, and cumquat (aka kumquat). Preferably, the signalling agent is one that is not known to be or implicated as being an irritant to the nasal mucosa, such as those selected from lemon, lime, cumquat (aka kumquat), and strawberry.

The signalling agent in may be present at 0.25% w/w to 2% w/w, preferably from 0.50% w/w to 2% w/w of the total weight of the composition. The biologically active agent makes up from 8% w/w to 9.75% w/w of the composition. Homogenised dry powder compositions of the invention consist of 90% w/w up to 99.75% w/w HPMC particles, depending on design.

Compositions of the invention are able to physically contain and/or disrupt the physiology of viruses selected from influenza viruses such as type A, H1N1, H5N1 and H3N2; coronaviruses, such as MERS-CoV, SARS-CoV, HCoV-229E, HCov-NL63, HCoV-OC43, CoV-HKU1, and 2019-nCoV (aka SARS-COV-2); and from bacteria such as Staphylococcus aureus, methicillin resistant Staphylococcus aureus, Haemophilus influenzae, Mycobacterium tuberculosis, Klebsiella pneumoniae, Pseudomonas aeruginosa, Acinetobacter baumannii, Enterococcus faecium, Candida albicans, Candida tropicalis and Enterobacter species.

In a second embodiment of the invention, there is provided a composition in the form of a dry homogenised powder consisting of
i) hydroxypropyl methylcellulose particles; and
ii) at least one chemical agent selected from signalling agents.

The dry homogenised powder (pHPMC) of the second embodiment of the invention has the same defined mean particle size as provided for, hereinabove.

Compositions according to the second embodiment of the invention contain a signalling agent or additive such as menthol, strawberry, mint, spearmint, peppermint, eucalyptus, lavender, and citrus, or any combination thereof. Examples of citrus may include lemon, lime, and cumquat (aka kumquat). Preferably, the signalling agent is one that is not known to be or implicated as being an irritant to the nasal mucosa, such as those selected from lemon, lime, cumquat (aka kumquat), and strawberry.

The signalling agent in the second embodiment of the invention makes up from 0.25% w/w to ≤10% w/w, preferably from 0.50% w/w to 5% w/w of the total weight of the composition. Homogenised dry powder compositions of the second embodiment of the invention consist of about 90% w/w up to 99.75% w/w HPMC particles as herein defined, depending on design. Preferably, compositions of the second embodiment of the invention contain HPMC particles as herein defined at >90, 91, 92, 93, 94, 95, 96, 97, 98 or 99% w/w of the composition again, depending on design.

The inclusion of such signalling agents in compositions of the invention is intended to provide the patient with sensory feedback upon use, in the form of a discernible sensation which allows the patient to recognize that administration has occurred and may aid the patient's recollection of administration. Thus, for the purposes of the present invention, a signalling agent is defined as one that primarily imparts an olfactory sensation and/or a taste sensation to the user.

The signalling agents may have other beneficial effects on the subject. Without the intention of being bound by theory certain formulations according to the present invention which include mint may have the effect of helping to dilate airways. This may be particularly beneficial when the formulations are used to treat patients suffering from asthma. Some patients, particularly those of a nervous disposition, tend to breathe in an irregular pattern. The administration of HPMC formulations including agents such as mint may also provide a feel-good factor which may be of help in restoring normal breathing patterns.

In a third embodiment of the invention, there is provided a composition consisting of
i) hydroxypropyl methylcellulose particles; and
ii) one or more biologically active agents selected from antiviral agents, antibacterial agents, and antiallergenic agents.

The biologically active agent consists of 2%, 3%, 4%, 5%, 6%, 7%, 8%, 9% or 10% w/w or any value therein between of the composition, depending on design. Homogenised dry powder compositions of the third embodiment of the invention consist of 90% w/w to 98% w/w pHPMC particles. Preferably, compositions of the third embodiment of the invention contain pHPMC particles as herein defined at >90, 91, 92, 93, 94, 95, 96, 97, or 98% w/w or any value therein between of the composition, depending on design.

Compositions of the third embodiment of the invention consist of one or more biologically active agents from therapeutic agents selected from pharmaceuticals, herbal and homeopathic agents. Suitable herbal and homeopathic agents are typically selected from those that have one or more of the following properties: antibacterial, antiviral, or anti-inflammatory function. A selection of biologically active agents of use in the third aspect of the invention is: St John's Wort, valerian extract, ginkgo biloba extract, vitamins A, E or C, garlic, one or more pro-biotics, ginger, ellagic acid, echinacea, Swedish flower pollen, black walnut hulls, lemongrass, wormwood, grapefruit seed extract, broccoli, digestive enzymes, hyaluronic acid, astragalus, rosehips, gentian, hypericum, horse chestnut, ginseng, green tea, phosphatidyl serine, phosphatidyl choline, pycnogenol, caffeine, quercitin, co-enzyme Q10, yarrow, tea tree, noni juice, lipase, fructo-oligosaccharide, inulin, black cumin, stabilised allicin, or any combination thereof.

Further suitable biologically active agents of use in the third embodiment of the invention are pharmaceuticals that show promise against disease caused by coronaviruses, such as severe acute respiratory syndrome (SARS), Covid 19 caused by SARS-COV 2 virus, Middle East Respiratory Syndrome (MERS), coronavirus E229E, and mutant strains thereof are selected from Type I (α, β) interferons (IFN), such as IFN-β, IFNβ-1b, Type II (γand Type III (λ) interferons, favipiravir,(aka favilovir, T-705, and Avigan) available from Fujifilm Toyama Chemical, remdesivir, ozeltamivir, zanamivir, ribavirin, lopinavir, combination of lopinavir-ritonavir and IFNβ-1b, monoclonal and (camel) polyclonal neutralising antibodies and macrolides, such as ivermectin, plant alkaloids such as colchicine and the like. A compound that shows potential for use against coronaviruses in general is K22, structural name (Z)-N-(3-(4-(4-bromophenyl)-4-hydroxypiperidin-1-yl)-3-oxo-1-phenylprop-1-en-2-yl)benzamide, available from ChemDiv (San Diego, Calif.: catalog number 4295-0370). This compound targets membrane-bound viral RNA synthesis and shows potent inhibition in diverse coronaviruses, including MERS virus. Further suitable biologically active agents include isolated griffithsin lectin proteins of about 121 amino acids extractable from a red algae seaweed such as Griffithsia, and biologically active, isolated antiviral analogues thereof, and natural seaweed extracts containing it (Journal of Virology 2010, O'Keefe, B.R et al ‘Broad Spectrum In Vitro Activity and In Vivo Efficacy of the Antiviral Protein Griffithsin against Emerging Viruses of the Family Coronaviridae’. Published online DOI: 10.1128/JVI.02322-09⁸; Marine Drugs 2019 October ; 17(10): 567, Choongho Lee ‘Griffithsin a highly potent Broad Spectrum Anti-Viral Lectin from Red Algae: From Discovery to Clinical Application’ Published online 2019 Oct. 6. doi: 10.3390/md17100567.

Preferred pharmaceuticals include remdesivir and ivermectin.

Typically, compositions of the invention provide for sustained release of biologically active agents as herein defined. Typically, the biologically active agent has a systemic effect upon nasal administration.

In certain embodiments of the present invention, the combination of the HPMC, signalling agent and biologically active agent is provided for sequential or simultaneous administration. The HPMC, signalling agent and biologically active agent may be included together in a single preparation. Alternatively, the HPMC, signalling agent and biologically active agent may be provided in separate preparations, for sequential administration. Where administration is sequential, the HPMC and/or signalling agent may be administered before or after the biologically active agent, or both. Similarly, the biologically active agent may be administered before or after the HPMC and/or signalling agent, or both.

Where the powdered HPMC and/or signalling agent are included in the same preparation as the biologically active agent, the preparation is preferably in the form of a powder. Where the powdered HPMC and/or signalling agent are included in a separate preparation to the biologically active agent, the HPMC is preferably in the form of a powder. The biologically active agent may, however, be in any form and is preferably in a form suitable for nasal administration, such as in the form of a powder, a liquid, a cream, or a gel.

In compositions of the invention, the powdered HPMC is present in a proportion of at least 90, 91, 92, 93, 94, 95, 96, 97, 98 or 99% w/w of the total weight of the composition depending on design.

In another embodiment of the present invention, the combination of the HPMC and the biologically active agent is provided for sequential or simultaneous administration. The HPMC and the biologically active agent may be included together in a single preparation. Alternatively, the HPMC and the biologically active agent may be provided in separate preparations, for sequential administration. Where administration is sequential, the HPMC may be administered before and/or after the biologically active agent. Alternatively, the biologically active agent may be administered before and/or after the HPMC.

Where the HPMC and the biologically active agent are included in separate preparations, the agent may be in any form suitable for intranasal administration, such as a powder, a liquid, a cream, or a gel.

According to an embodiment of the present invention, a kit is provided, comprising an HPMC powder composition, and a signalling agent as herein defined. Such kits are aimed at being used prophylactically or curatively to protect against viral attack from viruses such as influenza viruses such as type A, H1N1, H5N1 and H3N2; coronaviruses, such as MERS-CoV, SARS-CoV, HCoV-229E, HCov-NL63, HCoV-0043, CoV-HKU1, and 2019-nCoV (aka SARS-COV-2); and from bacteria such as Staphylococcus aureus, methicillin resistant Staphylococcus aureus, Haemophilus influenzae, Mycobacterium tuberculosis, Klebsiella pneumoniae, Pseudomonas aeruginosa, Acinetobacter baumannil, Enterococcus faecium, Candida albicans, Candida tropicalis, Enterobacter species and the like.

As alluded to herein, a signalling agent additive may be selected from menthol, mint, spearmint, peppermint, eucalyptus, lavender, citrus, strawberry or any combination thereof. Preferably, the signalling agent is one that is not known to be or implicated as being an irritant, such as those selected from strawberry, lemon, lime, cumquat (aka kumquat), or other citrus source.

The signalling agent makes up from 0.25% to 0%, preferably from 0.50% to 5% of the total weight of the applied composition.

According to a fourth embodiment of the present invention there is provided a kit comprising a HPMC powder composition, a signalling agent and a biologically active agent for simultaneous or sequential administration. The signalling agent makes up from 0.25% to 0%, preferably from 0.50% to 5% of the total weight of the composition. Such kits are also aimed at being used prophylactically or curatively to protect against viral attack from viruses such as influenza A viruses, for example, H1N1,H5N1, and H3N2; coronaviruses, such as MERS-CoV, SARS-CoV, HCoV-229E, HCov-NL63, HCoV-OC43, CoV-HKU1, and 2019-nCoV (aka SARS-COV-2); and from bacteria such as Staphylococcus aureus, methicillin resistant Staphylococcus aureus, Haemophilus influenzae, Mycobacterium tuberculosis, Klebsiella pneumoniae, Pseudomonas aeruginosa, Acinetobacter baumannii, Enterococcus faecium, Candida albicans, Candida tropicalis, Enterobacter species and the like.

A suitable biologically active agent may be selected from St John's Wort, valerian extract, ginkgo biloba extract, vitamins A, E or C, garlic, one or more pro-biotics, ginger, ellagic acid, echinacea, Swedish flower pollen, black walnut hulls, lemongrass, wormwood, grapefruit seed extract, broccoli, digestive enzymes, hyaluronic acid, astragalus, rosehips, gentian, hypericum, horse chestnut, ginseng, green tea, phosphatidyl serine, phosphatidyl choline, pycnogenol, caffeine, quercitin, co-enzyme Q10, yarrow, tea tree, noni juice, lipase, fructo-oligosaccharide, inulin, black cumin, stabilised allicin, or any combination thereof.

Further suitable biologically active agents that show promise against disease caused by coronaviruses, such as severe acute respiratory syndrome (SARS) and Middle East Respiratory Syndrome (MERS) include Type I (α, β) interferons (IFN), such as IFN-β, IFNβ-1b, Type II (γ) and Type III (λ) interferons, remdesivir, ozeltamivir, zanamivir, ribavirin, lopinavir, combination of lopinavir-ritonavir and IFNβ-1b, monoclonal and (camel) polyclonal neutralising antibodies and macrolides, such as ivermectin, plant alkaloids, such as colchicine, and the like. A compound that shows potential for use against coronaviruses in general is K22, structural name (Z)-N-(3-(4-(4-bromophenyl)-4-hydroxypiperidin-1-yl)-3-oxo-1-phenylprop-1-en-2-yl)benzamide, available from ChemDiv (San Diego, Calif.: catalog number 4295-0370). This compound targets membrane-bound viral RNA synthesis and shows potent inhibition in diverse coronaviruses, including MERS virus. Further suitable biologically active agents include isolated griffithsin lectin proteins of about 121 amino acids extractable from a red algae seaweed such as Griffithsia, and biologically active, isolated antiviral analogues thereof, and natural seaweed extracts containing it (Journal of Virology 2010, O'Keefe, B.R et al ‘Broad Spectrum In Vitro Activity and In Vivo Efficacy of the Antiviral Protein Griffithsin against Emerging Viruses of the Family Coronaviridae’. Published online DOI: 10.1128/JVI.02322-09; Marine Drugs 2019 October; 17(10): 567, Choongho Lee ‘Griffithsin a highly potent Broad Spectrum Anti-Viral Lectin from Red Algae: From Discovery to Clinical Application’ Published online 2019 Oct. 6. doi: 10.3390/md17100567.

Again, the signalling agent or additive may be selected from menthol, strawberry, mint, spearmint, peppermint, eucalyptus, lavender, citrus, or any combination thereof. Preferably, the signalling agent is one that is not known to be or implicated as being an irritant, such as those selected from lemon, lime, cumquat (aka kumquat), or other citrus source.

The signalling agent makes up from 0.25% to ≤10%, preferably from 0.50% to 3% of the total weight of the applied composition.

The powder compositions of the invention do not include any other additives or molecular components because such additives may interfere with the ability of inventive compositions to form gels on application to the nasal mucosa. Such additives deleterious to the formation of gels in the nasal passages include citric acid in combination with sodium citrate and benzalkonium chloride. Furthermore, other additives or components which are often used in intranasal compositions, such as other dry powders or solutions can cause irritation or affect ciliary movement, for example, solvents, such as propylene glycol, absorption enhancers, such as cyclodextrins or glycosides, or muco-adhesives such as chitosan. The use of such additives can be undesirable, as they can cause discomfort and interfere with the normal functioning of the nose, which can adversely affect breathing.

Powder ingredients may be blended together using a ribbon blender, or similar type of blender for approximately 15 to 20 minutes. The time of mixing is dependent upon the moisture content and compatibility of the powders. Ingredients preferably have a moisture content of less than 5% immediately after blending as checked with the United States Pharmacopeia and National Formulary (USP/NF) loss on drying method.

Devices which are suitable for dispensing the compositions according to the present invention are disclosed in, for example, EP1368090B1 and EP3183022B1, the teaching of which is incorporated herein in its entirety. The bottles disclosed therein use a very simple mechanism for restricting the amount of powder which is dispensed. Whilst the amount of powdered cellulose delivered to the nasal tract in order to enhance natural mucus does not have to be precisely controlled, the administration of too much powder could potentially cause an uncomfortable blockage of the nasal tract and may even result in difficulty in breathing through the nose.

The compositions according to the present invention are preferably administered in amounts of between about 1 mg and about 10 mg per nostril. Preferably, the dose is between about 2.5 mg to about 7.5 mg, between 3 mg and about 7 mg, between about 4 mg and about 6 mg, or about 5 mg.

In a fifth embodiment of the invention there is provided dry, free-flowing pHPMC particles per se (that is, before addition of other dry powder components forming compositions of the invention) for use in the invention that have an irregular size and shape as shown herein and that possess a mean particle size of 118 μm when freshly prepared. Under storage conditions, moisture from the air may be absorbed by the particles causing them to swell by up to 14% but still remain in free-flowing powder form. Particles that have been stored may assume a mean particle size of up to about 134 μm. Thus particles of HPMC per se may have a mean particle size of from about 110 μm to 140 μm, preferably from about 115 μm to about 135 μm, more preferably from 118 μm to 134 μm depending on moisture uptake.

As a sixth embodiment of the invention, there is provided a composition in homogenised dry powder form consisting of hydroxypropyl methylcellulose particles, and at least one chemical agent in particle form selected from signalling agents, wherein the homogenised powder comprises particles having a mean particle size of ≥20 μm to ≤500μm for use in treating or containing respiratory disease in a mammal such as a human being, said disease being caused by one or more of a virus, airborne allergens, such as plant pollens and house dust mite, and/or airborne pollution particles, such as PM_{2.5} and PM₁₀. Such compositions may be used to treat respiratory disease caused by viruses, such as coronaviruses selected from MERS-CoV, SARS-CoV, and 2019-nCov (aka SARS-COV-2).

A composition of this embodiment of the invention preferably consists of powdered HPMC particles wherein the mean particle size of the particles is in the range of 60 to 150 μm, preferably from 80 to 125 μm. more preferably 86 μm +/− 15 μm; a signalling agent selected from the group: mint, spearmint, peppermint, eucalyptus, lavender, citrus, or any combination thereof. Preferably, the signalling agent is selected from citrus, lemon, lime, cumquat (aka kumquat), or any combination thereof. The signalling agent makes up from 0.25% to ≤10% of the total weight of the composition.

As a further embodiment of the invention, there is provided a composition in homogenised dry powder form consisting of hydroxypropyl methylcellulose particles, and at least one chemical agent in particle form selected from signalling agents, and one or more biologically active agents wherein the homogenised powder comprises particles having a mean particle size of ≥20 μm to ≤500 μm, preferably of ≥60 to ≤150 μm, and more preferably from ≥80 to ≤125 μm, for example, 86 μm +/− 15 μm for use as a medicament for respiratory disease in a mammal, such as a human being, said disease being caused by one or more of a virus, airborne allergens, such as plant pollens and house dust mite, and/or airborne pollution particles, such as PM2.5 and PM₁₀. Such compositions may be used to treat respiratory disease caused by viruses, such as coronaviruses selected from MERS-CoV, SARS-CoV and 2019-nCov (aka SARS-COV-2). Definitions relating to all homogenised dry powder components and amounts thereof in this aspect of the invention are as defined hereinbefore.

As a further embodiment of the invention there is provided a composition as herein defined, wherein the said composition is for use as a nasally administered medicament.

As a yet further embodiment of the invention there is provided a composition as herein defined, wherein the said composition is for use in treating covid-19 disease.

As a still further embodiment of the invention there is provided a composition as herein defined, wherein the said composition is for use in prophylaxis of covid-19 disease.

Naturally, the skilled artisan will appreciate that all compositional embodiments of the invention detailed herein are for delivery to the nasal mucosa via insufflation through the nose.

There is also provided a method of making a powdered composition for use as a medicament for treating covid-19 disease comprising:
1) adding signalling agent powder to hydroxypropyl methylcellulose powder;
2) diffusively blending the two ingredients of 1) in a blending machine; and
3) optionally adding powdered biologically active agent and further blending.

There now follow examples and Figures illustrating the invention. It is to be understood that the teaching of the examples and figures is not to be construed as limiting the invention in any way.

### FIGURES

FIG. 1: Cumulative volume (average) of successive powder batches over 2 years. It was analyzed by Particle size analyzer (Beckman Coulter LS Particle size analyzer).

FIG. 2: Particles constituting HPMC in scanning electron microscope (100× magnification) indicates morphology as a key factor in deposition in the airway.

FIG. 3: Step Wise Procedure for Preparation of samples and Testing pHPMC of the invention and a comparison with an Hypromellose (cHPMC) powder of a competitor company against Der p 1 Allergen Using Agar Diffusion Method (In VITRO)

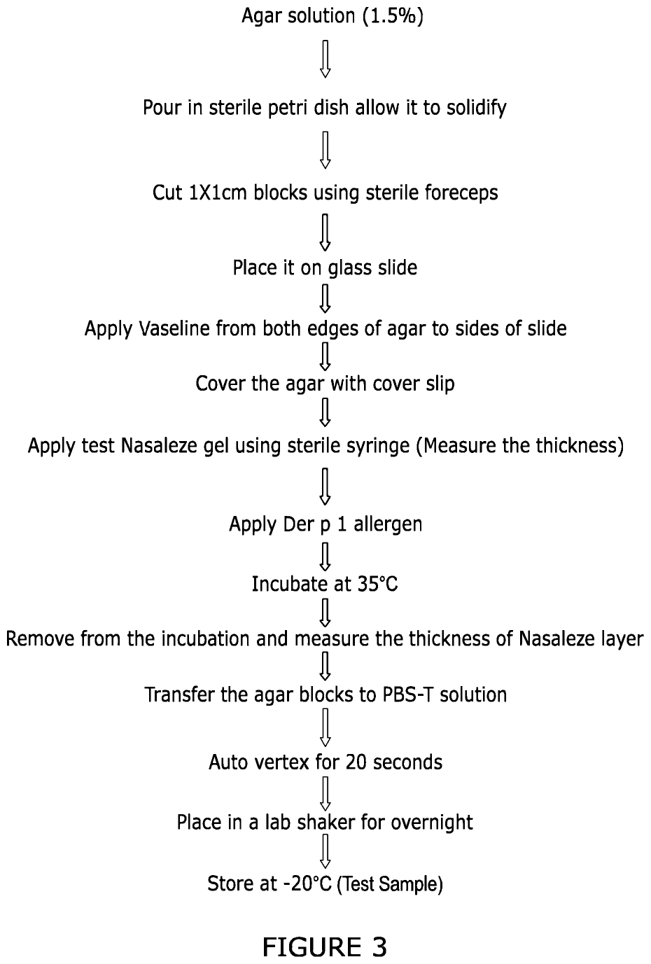

Referring to the protocol of FIG. 3:

*For Controlled Sample: Follow the above same procedure without adding sample layer and agar block with extra thickness.

#For Reference Sample: Follow the above same procedure without adding HPMC or Hypromellose layer and Der p 1

For baseline measurement add 20 μl Der p 1 antigen to 0.5ml of PBS-T Solution and follow above last 3 steps Followed by ELISA measurements.

### EXPERIMENTAL SECTION

### Section 1

### Physico-Chemical Characterization of Powdered HPMC (HPMC) Supporting the Safety Profile

The HPMC per se has been fully characterized. The physical and biochemical properties of HPMC, which is an inert natural product, do not give ground for safety concerns. Its favourable safety profile has been supported in all clinical studies performed so far, in none of which serious and/or severe adverse events have been reported (Popov T A, Aberg N, Emberlin J, et al. Methyl-cellulose powder for prevention and management of nasal symptoms. Expert review of respiratory medicine. Nov 2017;11(11):885-892)^{5,7}. A single ex-vivo study suggests that higher doses of cellulose powder may have a negative effect on the viability of the nasal epithelium and on its ciliary beat frequency (Zhou M, Zuo K J, Xu Z F, et al. Effect of Cellulose Powder on Human Nasal Epithelial Cell Activity and Ciliary Beat Frequency. International archives of allergy and immunology. 2019;178(3):229-237)⁹. Still, as it is intended for use by insufflation into the nose, we undertook a thorough characterization of the compound and performed a toxicology study in rats.

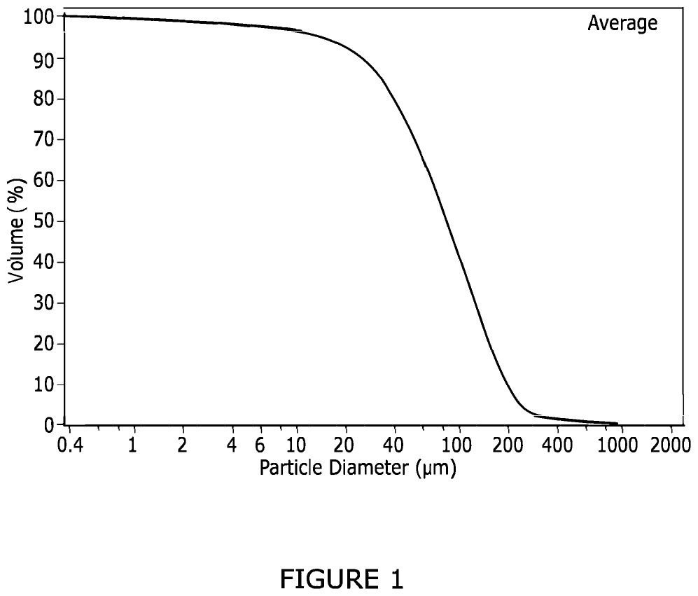

We routinely assayed HPMC batches by laser diffraction technology to obtain average particle size. The particle size distribution has been measured and 99.4% of particles have fallen within the 5 to 500 μm diameter range, with a mean particle size of 118 μm (FIG. 1).

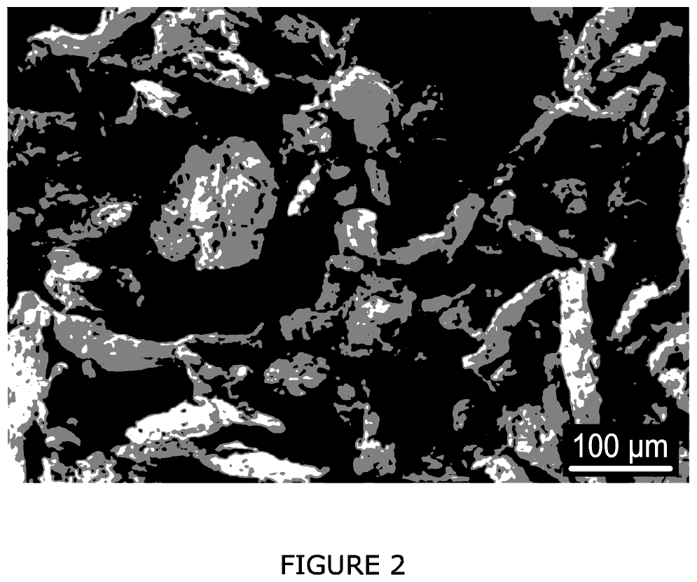

Particle count and mass distribution were measured in triplicate using a Grimm 1.109 laser particle counter connected to the software Grimm Dust Monitor 3.20. Test-retest reliability was assessed using correlation analysis: it produced a Pearson coefficient of 0.998 and 0.985. The actual particle mass and count distributions proved to be variable with a mean for all particle sizes of 6,095.0 μg/m³ and a standard deviation of 4,709.9 μg/m³ for mass distribution (75.4% of the mean), and a mean of 619,135,967 counts/m³ with a standard deviation of 330,964,124 counts/m³ for count distribution (57.5% of the mean). The particle size distribution of HPMC is significantly skewed towards larger particles. The pattern of distribution of the HPMC particles depends on the practical delivery methods utilized to deliver the powder, on their morphology and swelling behaviour due to the hygroscopic nature of cellulose (Telko M J, Hickey A J. Dry powder inhaler formulation. Respiratory care. Sep 2005;50(9):1209-1227)¹⁰. Particles of HPMC are characterized by uneven shape and surface which might affect nasal deposition (FIG. 2). The rough structure of the particles improves swelling by increased contact area which results in more efficient and faster swelling in the nose (Diethart B. The use of inert Hydoxypropyl methylcellulose powder as a remedy for allergic rhinitis. [Chpt 10 “The effect of HPMC application on human nasal cells”.]: University of Coventry in collaboration with University of Worcester; 2009).

Other determinants of the deposition in the nasal cavity are shape, density, potential electric charges, individual breathing patterns and the airflow rate. Particles larger than 5 μm are deposited in the nasopharynx, while particle sizes between 1 and 5 μm, if actively inhaled, can be deposited on the walls of the trachea and bronchial tree. Particles deposited in the nose, and in the tracheo-bronchial airway are trapped in the mucous lining, travel along with it to the pharynx and are swallowed. Only particle sizes below 1 micron could potentially reach the alveoli. In our study only 0.63% of the particles were of less than 5 μm diameter, and no particles of less than 1.9 μm were detected. In other words, essentially none of the HPMC particles would reach the alveoli, therefore the whole amount can be considered as swallowed. This kind of spectrum of particle sizes favours the targeted deposition in the nasal cavity in achieving maximal local effect in protecting the mucosa from allergens in allergic rhinitis and any irritants or infectious agents in non-allergic rhinitis.

Particle swelling begins immediately upon contact with moisture in the nasal tract and the powder also absorbs moisture from nasal air causing a growth in diameter. It is thought that this leads to augmented deposition within the nose which increases in efficiency with increasing particle size. These unique properties offer an explanation as to the role HPMC may play in quickly resolving symptoms of seasonal allergic rhinitis. Overall, HPMC is a remarkably safe material when given orally in gram quantities, and the use of Nasaleze in milligram amounts for insufflation in the nose does not present a recognizable risk. Based on the no-observed-adverse-effect level (NOAEL) of 5000 mg/kg body weight/day from a 90-day feeding study in rats, a tolerable intake for ingestion of HPMC by humans of 5 mg/kg body weight/day is accepted, which is more than 100-fold greater than the estimated current consumption of 0.047 mg/kg body weight/day (Burdock Ga. Safety assessment of hydroxypropyl methylcellulose as a food ingredient. Food and chemical toxicology: an international journal published for the British Industrial Biological Research Association. December 2007; 45(12):2341-2351)¹¹. No studies of genotoxicity, or reproductive toxicity have been identified, but the chemistry of the materials, their recognized safety in food use and lack of toxicity in feeding trials, does not suggest that further studies are necessary.

In conclusion, the in vitro studies support the capacity of HPMC to form gel upon contact with moisture, which provides a reliable barrier to airborne allergens and particulate matter. A study in rats also shows that insufflation of rather high doses of HPMC through their mouths does not affect the lungs, heart and livers of the animals. In clinical practice HPMC is not supposed to be inhaled into the lower airways: the cited animal study provides an additional safeguard that even if this happens unintentionally, no harmful consequences are to be expected.

### Expert Commentary

Precluding the contact between the nasal mucosa and the harmful agents in the ambient environment which attack it (allergens, irritants, microorganisms) is the simplest and most natural approach to prevent triggering inflammatory events in the airways and the ensuing clinical symptoms. This approach is referred to as “barrier-enforcing measures” and may be viewed as a means to achieve allergen avoidance (Andersson M, Greiff L, Ojeda P, Wollmer P. Barrier-enforcing measures as treatment principle in allergic rhinitis: a systematic review. Current medical research and opinion. June 2014; 30(6):1131-1137)¹².

Ideally, if implemented properly, this strategy could make the use of any other therapeutic action unnecessary. Attempts have been made to use different substances as barrier enhancers: white vaseline, pollen blocker cream, lipid-based ointment, microemulsion, liposomal formulation, seawater gel.

Many of the listed approaches could not withstand the test of time and have been abandoned. Microcrystalline powder Hydroxy-propyl-methylcellulose (HPMC) has been developed into a patented medical device and licensed in the management of allergic rhinitis (Product general information available at https://www.nasaleze.com/) Its clinical efficacy and real-world effectiveness have been proven in dozens of studies. There had been open questions along the road, which have been taken into consideration and tested in laboratory, in vitro and ex vivo studies. The present overview provides previously unpublished data, which can be of use to the medical and patients communities as a basis for wider application of a natural product for prevention and treatment of airway diseases.

Key Issues
HPMC is a cellulose derivative powder with a patented drug delivery system.
HPMC insufflated in the nose releases a spectrum of particles 99.4% of which fall within the 5 to 500 μm diameter range.
HPMC particles are highly hygrosopic and have a rough shape and surfaces resulting in fast swelling and gel formation when insufflated in the nose.
The HPMC gel layer sets a barrier and prevents the contact of the nasal mucosa with pollen, house dust mite allergens and particulate matter 2.5 μm (PM_{2.5}) (avoidance effect).
In addition to the theoretical arguments and the long-time experience with cellulose derivatives, a study in rats demonstrated that HPMC does not deposit in the lungs and does not cause adverse systemic effects.

### Section 2

Particle Size Description Before and After Homogenous Mixing of Cellulose with Signalling Agents

### IOM915K HPMC Cellulose Powder Particle Size Definition

Plain HPMC designated as IOM915K is a polydisperse powder specifically targeted at the extrathoracic airways. Over 96% of the IOM915K powder when instilled into the nasal cavity is available for gel formation which begins immediately.

Initial particle size measurements indicated that plain cellulose powder varied between 2 microns and 478.50 microns with a mean particle size of about 118 μm.The introduction of a signalling agent designed to allow the end user to determine when an effective dose was instilled was introduced in 2006 following reports that it was difficult to be certain that a dose had been instilled in early clinical trial work (Josling P, Steadman S, Use of cellulose powder for the treatment of seasonal allergic rhinitis, Adv Therapy 20, 213-219 , 2003¹³; and Emberlin J C and Lewis R A, A double blind placebo controlled trial of inert cellulose for the relief of hay fever in adults, Current Med Research and Opinion, 22, 275-285, 2006)¹⁴.

This meant that powder homogenisation and controlled mixing procedures were adopted as follows.

### Homogenisation of Powders

We use IOM915K HPMC powder and mix it with our established signalling agents which include lemon, mint, garlic and strawberry under standard operating procedure.

QMS Procedure 4 revision 11 dated 10 Jul. 2018. Powders are routinely assayed for moisture content, density and particle size as well as the standard microbial analyses. Mixing of powders is completed using a V blender for a total of 15 minutes for each mixture. This follows our protocol for storage of powders, preparation for mixing, calculation of mixing proportions and use of an industrial grade V blender machine (V100, model number A39525-2, sourced from Key Packaging Machinery Limited).

The V-Blender is made of two hollow cylindrical shells joined at an angle of 75° to 90°. The blender container is mounted on trunnions to allow it to tumble. As the V-blender tumbles, the material continuously splits and recombines, with the mixing occurring as the material free-falls randomly inside the vessel. The repetitive converging and diverging motion of material combined with increased frictional contact between the material and the vessel's long, straight sides result in gentle yet homogenous blending.

The primary mechanism of blending in a V-Blender is diffusion. Diffusion blending is characterized by small scale random motion of solid particles. Blender movements increase the mobility of the individual particles and thus promote diffusive blending. Diffusion blending occurs where the particles are distributed over a freshly developed interface. In the absence of segregating effects, the diffusive blending will in time lead to a high degree of homogeneity. V-Blenders are therefore preferred when precise blend formulations are required. They are also well suited for applications where some ingredients may be as low as five percent of the total blend size, as is the case with our homogenisation between IOM915K and any of our signalling agents that are present at under 5% of the total blended mixture. Normal blend times are typically 15 minutes to ensure complete homogenisation of our powders.

### IOM915K Plus a Signalling Agent Particle Size Definition

Following powder homogenisation the particle sizes alter markedly from the plain IOM915K. Inevitably when mixing a percentage of the larger particulate will be reduced in size as they are broken up in the V blender as they are mixed with our signalling agents. Similarly a small proportion of the smaller particulates under 5 microns will clump together to form larger particles. Analysis of particle sizes indicates that the powder mix is from 4 to 395 microns with a mean of 86.2 μm.

Further work has also indicated that over time i.e. a period of several months the overall particle size mean tends to increase.

HPMC mixtures increase in size by approximately 14% during storage at ambient temperature. It is therefore postulated that our powders absorb moisture from the air and grow in diameter causing them under instillation into the nasal cavity to deposit themselves in a higher position within the respiratory tract. This could lead to an augmented deposition within the nose which increases in efficiency with increasing particle size.

From the large clinical trial database that now affords our Nasaleze family of extracts which contain IOM915K cellulose and patented formulations of lemon, mint, strawberry and garlic we have shown that no small particulate reaches the lungs or brain and that our mean particle size of 86.2 μm together with the associated mass of these particulates allow for very effective control of symptoms in persistent allergic rhinitis and in the removal of pathogens including pollen, virus, bacteria, fungus and environmental toxins such as PM_{2.5} and PM₁₀.

### Section 3

### Section 3(a)

Determination of the Preventative and Treatment Capabilities of pHPMC Powder Formulated with Mint as a Signalling Agent and Wild Garlic Extract against Coronavirus 229E.
1.0 Aim

To determine the anti-viral efficacy of Nasaleze® powder (mean particle size of about 82 μm) against Human coronavirus 229E (CoV 229E).

5% w/w European Wild Garlic Extract was obtained from Pfannenschmidt GmbH. Hamburg, Germany

93% w/w Nasaleze® powder from Nasaleze Limited (on site)
2.0 Materials and Methods
2.1 Test Organisms

### Cell Types:

Medical Research Council human fibroblast cell line 5 [MRC-5 (ATCC® CCL-171)]

Virus: Human coronavirus 229E (CoV 229E) (ATCC® VR-740)
2.2 Test Agents

Test agent used in this study is shown in Table 1.

TABLE 1
Test agent used throughout the study.

| Test agent | Test agent format |
|---|---|
| pHPMC | HPMC powder containing European |
| | Wild Garlic |

2.3 Equipment and Media

### Equipment:

Class II biosafety cabinet-BioMAT, ThermoFisher Scientific, UK Vortex-Grant Instruments, UK

UKAS calibrated multichannel pipette (P300)-Gilson®, UK UKAS calibrated multichannel pipette (P20)-Gilson®, UK

UKAS calibrated pipettes (0.5-1000 μL range)-Proline® Plus, UK 96-well plates-ThermoFisher Scientific, UK

CO₂ Incubator-Thermo Scientific, UK

Tissue culture flasks-Nunc, ThermoFisher Scientific, UK Olympus CK2 Inverted microscope-KeyMed, UK

VWB2 Water bath-VWR, UK

Vacuboy Aspirator-INTEGRA, UK

### Media:

Phosphate buffered saline (PBS)-Gibco™, UK

Penicillin-streptomycin-ThermoFisher Scientific, UK

Eagle's Minimum Essential Medium (EMEM)-ATCC®, UK Dulbecco's Phosphate buffered saline (DPBS) Gibco™, UK

Fetal Bovine Serum (FBS) -Gibco™, USA

Trypsin-EDTA-Gibco™, UK

Trypan blue-Sigma-Aldrich, UK
2.4 Method
2.4.1 Cell Maintenance and Assay Set-Up

MRC-5 cells were used as the host cell line for human coronavirus 229E (CoV 229E) propagation. MRC-5 cells were maintained in Eagle's Minimum Essential Medium (EMEM) supplemented with 20% Foetal Bovine Serum (FBS) and 1% penicillin-streptomycin (complete EMEM) at 37±2° C. and 5% CO₂. In preparation for the cytotoxicity screening and anti-viral assays, MRC-5 cells were seeded into 24 well plates at 1.0×10⁵ cells/mL and incubated at 37±2° C. and 5% CO₂ for 24 hours, or until they reached 80-90% confluency. In preparation for tissue culture infectivity dose 50 (TCID₅₀) testing, MRC-5 cells were seeded into 96 well plates at 2×10⁵ cellsmL⁻¹ and incubated at 37±2° C. and 5% CO₂ for 24 hours.
2.4.2 Phase 1: Cytotoxicity Screen of Nasal Spray Formulation

Nasaleze® HPMC powder was diluted to 3.2 mg/0.1 mL, 6.4 mg/0.1 mL and 12.8 mg/0.1 mL in EMEM supplemented with 2% FBS and 1% penicillin-streptomycin (assay medium). Complete EMEM was aspirated from the test plates and 100 μL of each test concentration was added to duplicate wells. Following a 10-minute incubation period at 20±2° C. an additional 400 μL of assay medium was added to the test wells. Plates were incubated for 24 hours at 37±2° C. and 5% CO₂. Following incubation, visual scoring was performed on a scale of 0 to 4 according to ISO 10993-5 guidelines (Table 2). Cytotoxic effects were assessed based on a variety of morphological changes to the MRC-5 cells such as cell rounding, detachment and cell lysis.

TABLE 2
Cytotoxicity visual scoring and reactivity classifications.

| Visual | Cells with cytotoxic effects | Reactivity |
|---|---|---|
| Score | (%) | classification |
| 0 | 0 | None |
| 1 | 0-20 | Slight |
| 2 | 20-50 | Mild |
| 3 | 50-70 | Moderate |
| 4 | 70-100 | Severe |

2.4.3 Phase 2: Assessment of the Preventative and Treatment Capabilities of Nasaleze® Powder

MRC-5 cells were treated with Nasaleze® powder according to two methods to determine the preventative and treatment capabilities of the formulation. The assays were performed in 24-well plates utilising duplicate wells for each experimental condition.
2.4.3.1 Preventative Treatment of MRC-5 Cells using Nasaleze® Powder before Infection with Human Coronavirus 229E

To assess the preventative capabilities of Nasaleze® powder against CoV 229E, MRC-5 cells were pre-treated with 3.2 mg of the formulation for 10 minutes before infection with CoV 229E multiplicity of infections (MOIs) of 1 (high dose) and 0.01 (low dose). Complete EMEM was aspirated from the test plates and washed once in Dulbecco's phosphate buffered saline (DPBS) before application of 3.2 mg Nasaleze® powder in 100 μL assay media. Following a 10 minute incubation at 20±2° C., cells were inoculated with 100 μL CoV 229E, pre-diluted to achieve the high and low MOI infection, and incubated at 35±2° C. and 5% CO₂ for 30 minutes. Infected cells were then supplemented with an additional 300 μL of assay medium and incubated at 35±2° C. and 5% CO₂ for four days. The cytopathic effect (CPE) of the virus on the MRC-5 cells was scored on days 2, 3 and 4 to the criteria described in Table 2. On days 3 and 4, 100 μL of media was harvested from each well to determine the viral titre before replacing with 100 μL of fresh assay medium. Harvested samples were stored at −80° C. until required for viral titre determination.
2.4.3.2 Treatment of Human Coronavirus 229E Infected MRC-5 Cells with Nasaleze® Powder

To assess the treatment capabilities of Nasaleze® powder against CoV 229E, MRC-5 cells were first infected with high and low CoV 229E MOls, 1 and 0.01 respectively, before treatment with the formulation. Complete EMEM was aspirated from the test plates and washed once in DPBS before being inoculated with 100 μL of pre-diluted CoV 229E to achieve high and low MOI infections and incubated at 35±2° C. and 5% CO₂ for 30 minutes. Following incubation, viral inoculum was removed and a 3.2 mg dose of Nasaleze® powder in 100 μL assay media was added to the cells and incubated for 10 minutes at 20±2° C. to allow the formation of the gel barrier. Treated cells were then supplemented with an additional 300 μL of assay medium and incubated at 35±2° C. and 5% CO₂ for four days. The CPE of the virus on the MRC-5 cells was scored on days 2, 3 and 4 to the criteria described in Table 2. On days 3 and 4, 100 μL of media was harvested from each well to determine the viral titre before replacing with another 100 μL of fresh assay medium. Harvested samples were stored at −80° C. until required for viral titre determination.
2.4.4 Viral Infectivity Quantification by TCID₅₀

To determine the viral titre of harvested samples, 10-fold serial dilutions were performed in assay medium. Medium was aspirated from the wells of the cell plate and cells were washed with DPBS. One hundred microlitres of each dilution of the samples were added to the corresponding test wells. Test plates were incubated at 35±2° C. and 5% CO₂ for 7 days. There were four replicate wells for each test condition. After incubation, viral CPE was determined using an Olympus CK2 inverted microscope. The viral titre was calculated using the Spearman-Kerber method.
3.0 Results
3.1 Phase 1: Cytotoxicity Screen

There was no observable cytotoxicity in MRC-5 cells exposed to Nasaleze® powder following a 24 hour contact time (Table 3). When visual scoring was performed, the gel barrier formed with Nasaleze® powder was visible on top of the cell monolayer. Additionally, a residue was visible on treated cells (data not shown).

TABLE 3
Cytotoxicity of Nasaleze ® powder using visual scoring.

| | Visual | Reactivity |
|---|---|---|
| Treatment | score | classification |
| Nasaleze ® | 0 | No cytotoxicity |

3.2 Preventative Treatment of MRC-5 Cells using Nasaleze® Powder before Infection with Coronavirus 229E
3.2.1. Cytopathic Effect of CoV 229E on MRC-5 Cells Pre-Treated with Nasaleze® Powder

Following a 2, 3 and 4 day incubation period, the CPE of the test plate was scored (Table 4-6). CPE was observed (visual data not shown). Duplicate cells treated with Nasaleze® powder with a high MOI of CoV 229E showed slight CPE on day 2 and severe CPE on days 3 and 4. Duplicate cells treated with Nasaleze® powder with a low MOI of CoV 229E showed no CPE on day 2 and moderate CPE on days 3 and 4.

TABLE 4
Cytopathic effect observed on day 2 of cells pre-treated
with Nasaleze ® powder before infection with
coronavirus 229E. MOI = multiplicity of infection.

| | | |
|---|---|---|
| Treatment | Nasaleze ® Powder | Negative Control |

| | | | | |
|---|---|---|---|---|
| MOI 1 | 2 | 2 | 3 | 2 |
| MOI 0.01 | 0 | 0 | 1 | 0 |
| No Virus | 0 | 0 | 0 | 0 |
| No Virus | 0 | 0 | 0 | 0 |

TABLE 5
Cytopathic effect observed on day 3 of cells pre-treated
with Nasaleze ® powder before infection with
coronavirus 229E. MOI = multiplicity of infection.

| | | |
|---|---|---|
| Treatment | Nasaleze ® Powder | Negative Control |

| | | | | |
|---|---|---|---|---|
| MOI 1 | 4 | 4 | 4 | 3 |
| MOI 0.01 | 3 | 3 | 2 | 2 |
| No Virus | 0 | 0 | 0 | 0 |
| No Virus | 0 | 0 | 0 | 0 |

TABLE 6
Cytopathic effect observed on day 4 of cells pre-treated
with Nasaleze ® powder before infection with
coronavirus 229E. MOI = multiplicity of infection.

| | | |
|---|---|---|
| Treatment | Nasaleze ® Powder | Negative Control |

| | | | | |
|---|---|---|---|---|
| MOI 1 | 4 | 4 | 4 | 4 |
| MOI 0.01 | 3 | 3 | 3 | 3 |
| No Virus | 1 | 1 | 0 | 0 |
| No Virus | 4 | 1 | 0 | 0 |

3.2.2 Viral Titration of Samples Pre-Treated with Nasaleze® Powder

Following a 3 and 4 day incubation period with a high MOI of CoV 229E the negative control resulted in an average viral titre of 5.82±0.35 Log10TCID₅₀/mL and 5.32±0.35 Log10TCID₅₀/mL, respectively. Pre-treatment of MRC-5 cells with Nasaleze® powder resulted in a 2.68 Log10TCID₅₀/mL and 2.55 Log10TCID₅₀/mL reduction in viral titre on day 3 and day 4 post-infection, respectively, when compared to the negative control (Table 7).

TABLE 7
Log TCID₅₀ and Log reduction values for human coronavirus 229E
(CoV 229E) following treatment with Nasaleze ® powder
before infection at a high multiplicity of infection and incubated
for 3 and 4 days. N/A = not applicable, SD = standard deviation.

| | Average Viable CoV 229E ± SD | | LogReduction | |
|---|---|---|---|---|
| | (Log₁₀TCID₅₀/mL) | | (Log₁₀TCID₅₀/mL) | |
| Product | Day 3 | Day 4 | Day 3 | Day 4 |
| Negative Control | 5.82 ± 0.35 | 5.32 ± 0.35 | N/A | N/A |
| Nasaleze ® | 3.14 ± 0.18 | 2.77 ± 0.53 | 2.68 | 2.55 |
| powder | | | | |

Following a 3 and 4 day incubation period with a low MOI of CoV 229E the negative control resulted in an average viral titre of 6.02±0.53 Log10TCID₅₀/mL and 5.39±0.18 Log10TCID₅/mL, respectively. Pre-treatment of MRC-5 cells with Nasaleze® powder resulted in a 1.70 Log10TCID₅₀/mL and 1.00 Log10TCID₅₀/mL reduction in viral titre on day 3 and day 4 post-infection, respectively, when compared to the negative control (Table 8).

TABLE 8
Log TCID₅₀ and Log reduction values for human coronavirus 229E
(CoV 229E) following treatment with Nasaleze ® powder
before infection at a low multiplicity of infection and incubated
for 3 and 4 days. N/A = not applicable, SD = standard deviation.

| | Average Viable CoV 229E ± SD | | Log Reduction | |
|---|---|---|---|---|
| | (Log₁₀TCID₅₀/mL) | | (Log₁₀TCID₅₀/mL) | |
| Product | Day 3 | Day 4 | Day 3 | Day 4 |
| Negative Control | 6.02 ± 0.53 | 5.39 ± 0.18 | N/A | N/A |
| Nasaleze ® | 4.32 ± 0.35 | 4.39 ± 0.18 | 1.70 | 1.00 |
| powder | | | | |

3.3 Treatment Capabilities of Nasaleze® Powder
3.3.1 Cytopathic Effect of CoV 229E on MRC-5 Cells Treated with Nasaleze® Powder after Viral Infection

Following a 2, 3 and 4 day incubation period, the CPE of the test plate was scored (Table 9-11). Representative images of the CPE observed are presented in FIG. B. Duplicate cells treated with Nasaleze® powder after infection with a high MOI of CoV 229E showed mild CPE on day 2 and severe CPE on days 3 and 4 post-infection. Duplicate cells treated with Nasaleze® powder after infection with a low MOI of CoV 229E showed no CPE on day 2 and moderate CPE on days 3 and 4 post-infection.

TABLE 9
Cytopathic effect observed on day 2 of cells treated
with Nasaleze ® powder after infection with
human coronavirus 229E. MOI = multiplicity of infection

| | | |
|---|---|---|
| Treatment | Nasaleze ® powder | Negative Control |

| | | | | |
|---|---|---|---|---|
| MOI 1 | 2 | 2 | 2 | 2 |
| MOI 0.01 | 0 | 0 | 0 | 1 |
| No Virus | 0 | 0 | 0 | 0 |
| No Virus | 0 | 0 | 0 | 0 |

TABLE 10
Cytopathic effect observed on day 3 of cells treated
with Nasaleze ® powder after infection with
human coronavirus 229E. MOI = multiplicity of infection.

| | | |
|---|---|---|
| Treatment | Nasaleze ® powder | Negative Control |

| | | | | |
|---|---|---|---|---|
| MOI 1 | 4 | 4 | 3 | 3 |
| MOI 0.01 | 3 | 3 | 3 | 3 |
| No Virus | 1 | 1 | 0 | 0 |
| No Virus | 0 | 0 | 0 | 0 |

TABLE 11
Cytopathic effect observed on day 4 of cells treated
with Nasaleze ® powder after infection with
human coronavirus 229E. MOI = multiplicity of infection.

| | | |
|---|---|---|
| Treatment | Nasaleze ® powder | Negative Control |

| | | | | |
|---|---|---|---|---|
| MOI 1 | 4 | 4 | 3 | 3 |
| MOI 0.01 | 3 | 3 | 3 | 3 |
| No Virus | 1 | 1 | 0 | 0 |
| No Virus | 0 | 0 | 0 | 0 |

3.3.2 Viral Titration of Samples Treated with Nasaleze® Powder after Viral Infection

Following a 3 and 4 day incubation period with a high MOI of CoV 229E the negative control resulted in an average viral titre of 5.82±0.35 Log10TCID₅₀/mL and 5.32±0.35 Log10TCID₅₀/mL, respectively. Treatment of MRC-5 cells with Nasaleze® powder after infection with a high MOI of CoV 229E resulted in a 1.07 Log10TCID₅₀/mL and 1.93 Log10TCID₅₀/mL reduction in viral titre on day 3 and day 4 post-infection, respectively, when compared to the negative control (Table 12).

TABLE 12
Log TCID₅₀ and Log reduction values for human coronavirus
229E (CoV 229E) following treatment with Nasaleze ® powder
after infection at a high MOI and incubated for 3 and 4
days. N/A = not applicable, SD = standard deviation.

| | Average Viable CoV 229E ± SD | | Log Reduction | |
|---|---|---|---|---|
| | (Log₁₀TCID₅₀/mL) | | (Log₁₀TCID₅₀/mL) | |
| Product | Day 3 | Day 4 | Day 3 | Day 4 |
| Negative Control | 5.82 ± 0.35 | 5.32 ± 0.35 | N/A | N/A |
| Nasaleze ® | 4.75 ± 0.00 | 3.39 ± 0.18 | 1.07 | 1.93 |
| powder | | | | |

Following a 3 and 4 day incubation period with a low MOI of CoV 229E the negative control resulted in an average viral titre of 6.50±0.00 Log10TCID₅₀/mL and 5.89±0.18 Log10TCID₅₀/mL, respectively. Treatment of MRC-5 cells with Nasaleze® powder after infection with a low MOI of CoV 229E resulted in a 0.75 Log10TCID₅₀/mL and 1.00 Log10TCID₅₀/mL reduction in viral titre on day 3 and day 4 post-infection, respectively, when compared to the negative control (Table 13).

TABLE 13
Log TCID₅₀ and Log reduction values for human coronavirus
229E (CoV 229E) following treatment with Nasaleze ® powder
after infection at a low MOI and incubated for 3 and 4
days. N/A = not applicable, SD = standard deviation.

| | Average Viable CoV 229E ± SD | | Log Reduction | |
|---|---|---|---|---|
| | (Log₁₀TCID₅₀/mL) | | (Log₁₀TCID₅₀/mL) | |
| Product | Day 3 | Day 4 | Day 3 | Day 4 |
| Negative Control | 6.50 ± 0.00 | 5.89 ± 0.18 | N/A | N/A |
| Nasaleze ® | 5.75 ± 0.00 | 4.89 ± 0.18 | 0.75 | 1.00 |
| powder | | | | |

4.0 Discussion

The dissemination of potentially pathogenic viruses increases infection risk in both healthy and immunocompromised individuals. Coronaviruses are enveloped, single stranded RNA viruses responsible for a variety of upper-respiratory tract illnesses in humans. These illnesses range from mild conditions such as the common cold to severe acute respiratory syndrome as seen in the recent COVID-19 pandemic. Coronaviruses are thought to be predominantly transmitted through respiratory droplets with some evidence to suggest the virus can remain active on fomites for several days. Interventions, both preventative and curative, are essential to slowing and/or stopping the spread of coronaviruses. The assessment of inventions against coronavirus surrogate strains allows for the safe evaluation of product efficacy. Coronavirus 229E is structurally and genetically similar to the Sars-CoV-2 virus.

Two approaches were taken to investigate the anti-viral efficacy of Nasaleze® powder. In the first arm of the study, MRC-5 cells were pre-treated with Nasaleze® powder before infection with high and low doses of CoV 229E. The second approach infected MRC-5 cells with a high and low dose of CoV 229E before treatment with Nasaleze® powder. Treatment with Nasaleze® powder yielded substantial reductions in viral titre in both experimental arms of the study indicating a high level of anti-viral potential.

### Section 3(b)

1.0 Aim

To assess the anti-viral efficacy of two nasal dry powder spray products against Human coronavirus 229E using a preventative and treatment-based approach.
2.0 Materials and Methods
2.1 Test Organisms
   Cell types:

MRC-5 (ATCC® CCL-171˜) Passage number3

Virus: Human coronavirus 229E (CoV 229E)

(ATCC® VR-740™)-Amplification number: 1
2.2 Test Agents

Test agents used in the study are listed in Table 1.

TABLE 1
Test agents used throughout the study.

| Test agent | Test agent format | Lot number |
|---|---|---|
| 1. REM | Powder | 001 |
| 2. IVER | Powder | 002 |

1. REM consists of remdesivir at a concentration of 8% w/w admixed evenly with 90% HPMC particles and 2% signalling agent.
2. IVER consists of ivermectin at a concentration of 8% w/w admixed evenly with 90% w/w HPMC particles and 2% signalling agent.
2.3 Equipment and Media

### Equipment:

Class II biosafety cabinet-BioMAT, ThermoFisher Scientific, UK Vortex-Grant Instruments, UK UKAS calibrated multichannel pipette (P300)-Gilson®, UK UKAS calibrated multichannel pipette (P20)-Gilson®, UK

UKAS calibrated pipettes (0.5-1000 μL range)-Proline® Plus, UK 96-well plates-ThermoFisher Scientific, UK

24-well plates-ThermoFisher Scientific, UK CO₂ Incubator BB-15-Thermo Scientific, UK

Tissue culture flasks-Nunc, ThermoFisher Scientific, UK Olympus CK2 Inverted microscope-KeyMed, UK

VWB2 Water bath-VWR, UK Vacuboy

Aspirator INTEGRA, UK

### Media:

Phosphate buffered saline (PBS)-Gibco™, UK Penicillin-streptomycin-ThermoFisher Scientific, UK Eagle's Minimum Essential Medium (EMEM) ATCC®, UK

Dulbecco's Phosphate buffered saline (DPBS) Gibco™, UK

Fetal Bovine Serum (FBS)-Gibco™, USA Trypsin-EDTA Gibco™, UK Trypan blue Sigma Aldrich, UK
2.4 Method
2.4.1 Cell Maintenance and Assay Set-Up

MRC-5 cells were used as the host cell line for Human coronavirus 229E propagation. MRC-5 cells were maintained in Eagle's Minimum Essential Medium (EMEM) supplemented with 20% Foetal Bovine Serum (FBS) and 1% penicillin-streptomycin (complete culture EMEM) at 37±2° C. and 5% CO₂. In preparation for the cytotoxicity screening and anti-viral assays, MRC-5 cells were seeded into 24 well plates and incubated at 37±2° C. and 5% CO₂ for 24 hours, or until they reached 80-90% confluency.
2.4.2 Phase 1: Cytotoxicity Screen of Nasal Spray Formulations

Test items were diluted to 3.2 mg/0.1 mL, in EMEM supplemented with 2% FBS and 1% penicillin-streptomycin (assay medium). Complete culture EMEM was aspirated from the test plates and 100 μL of each test concentration was added to duplicate wells. Following a 10-minute incubation period at 20±2° C. an additional 400 μL of assay medium was added to the test wells. Plates were incubated for 24 hours at 37±±2═ C. and 5% CO₂. Following incubation, visual scoring was performed on a scale of 0 to 4 according to ISO 10993-5 guidelines (Table 2). Cytotoxic effects were assessed based on a variety of morphological changes to the MRC-5 cells such as cell rounding, detachment and cell lysis.

TABLE 2
Cytotoxicity visual scoring and reactivity classifications.

| Visual | Cells with cytotoxic effects | Reactivity |
|---|---|---|
| Score | (%) | classification |
| 0 | 0 | None |
| 1 | 0-20 | Slight |
| 2 | 20-50 | Mild |
| 3 | 50-70 | Moderate |
| 4 | 70-100 | Severe |

2.4.3 Phase 2: The Anti-Viral Efficacy of Two Nasal Spray Formulations against Human Coronavirus 229E using a Preventative- and Treatment-Based Approach

MRC-5 cells were treated with the nasal spray formulations according to two methods to determine the preventative and treatment capabilities of the formulation. The assays were performed in 24-well plates utilising duplicate wells for each experimental condition.
2.4.3.1 Preventative Treatment of MRC-5 Cells using Two Nasal Spray Formulations before Infection with Human Coronavirus 229E

To assess the prevention capabilities of the nasal sprays against Human coronavirus 229E, MRC-5 cells were pre-treated with 3.2 mg/0.1 mL of each formulation for 10 minutes before infection. Complete EMEM was aspirated from the test plates and washed once in Dulbecco's phosphate buffered saline (DPBS) and 3.2 mg of test powder in 100 μL of assay medium was applied. Following a 10 minute incubation at 20±2° C., cells were inoculated with 100 μL
2.4.3.2 Preventative Treatment of MRC-5 Cells using Two Nasal Spray Formulations before Infection with Human Coronavirus 229E

To assess the prevention capabilities of the nasal sprays against Human coronavirus 229E, MRC-5 cells were pre—treated with 3.2 mg/0.1 mL of each formulation for 10 minutes before infection. Complete EMEM was aspirated from the test plates and washed once in Dulbecco's phosphate buffered saline (DPBS) and 3.2 mg of test powder in 100 μL of assay medium was applied. Following a 10 minute incubation at 20±2° C., cells were inoculated with 100 μL Human coronavirus 229E, pre-diluted to achieve the high (0.3) and low (0.01) multiplicity of
2.4.3.3 Preventative Treatment of MRC-5 Cells using Two Nasal Spray Formulations before Infection with Human Coronavirus 229E

To assess the prevention capabilities of the nasal sprays against Human coronavirus 229E, MRC-5 cells were pre-treated with 3.2 mg/0.1 mL of each formulation for 10 minutes before infection. Complete EMEM was aspirated from the test plates and washed once in Dulbecco's phosphate buffered saline (DPBS) and 3.2 mg of test powder in 100 μL of assay medium was applied. Following a 10 minute incubation at 20±2° C., cells were inoculated with 100 μL Human coronavirus 229E, pre-diluted to achieve the high (0.3) and low (0.01) multiplicity of infections (MOI). Samples were incubated at 35±2° C. and 5% CO₂ for 30 minutes. Infected cells were then supplemented with an additional 300 μL of assay medium and incubated at 35±2° C. and 5% CO₂ for four days. On days 2, 3 and 4, 100 μL of media was harvested from each well to determine the viral titre. A 100 μL aliquot of fresh assay medium was applied to the cells following each harvest. Harvested samples were stored at −80° C. until required for viral titre determination by TCID₅₀. The viral titre was calculated using the Spearman-Karber method.
2.4.3.4 Treatment of Human Coronavirus 229E Infected MRC-5 Cells with Two Nasal Spray Formulations

To assess the treatment capabilities of the nasal sprays against Human coronavirus 229E, MRC-5 cells were first infected with high and low Human coronavirus 229E MOIs, 0.3 and 0.01 respectively, before treatment with each of the two formulations. Complete EMEM was aspirated from the test plates and washed once in DPBS. Samples were inoculated with 100 μL of pre-diluted Human coronavirus 229E to achieve high and low MOI infections and incubated at 35±2° C. and 5% CO₂ for 30 minutes. Following incubation, viral inoculum was removed and a 3.2 mg dose of test powder in 100 μL assay media was added to the cells and incubated for 10 minutes at 20±2° C. to allow the formation of the gel barrier. Treated cells were then supplemented with an additional 300 μL of assay medium and incubated at 35±2° C. and 5% CO₂ for four days. On days 2, 3 and 4, 100 μL of media was harvested from each well to determine the viral titre. A 100 μL aliquot of fresh assay medium was applied to the cells following each harvest. Harvested samples were stored at −80° C. until required for viral titre determination by TCID₅₀. The viral titre was calculated using the Spearman-Kärber method.
3.0 Results
3.1 Phase 1: Cytotoxicity Screen of Two Nasal Spray Formulations There was no observable cytotoxicity in MRC-5 cells exposed to the nasal sprays following a 24 hour contact time (Table 3).

TABLE 3
Cytotoxicity of the nasal spray formulations using visual scoring.

| Test | Visual | Reactivity |
|---|---|---|
| agent | score | classification |
| 1. REM | 0 | No cytotoxicity |
| 2. IVER | 0 | No cytotoxicity |

3.2 Phase 2: The Anti-Viral Efficacy of Two Nasal Spray Formulations against Human Coronavirus 229E using a Preventative and Treatment-Based Approach.
3.2.1 Preventative Treatment of MRC-5 Cells using the Nasal Spray Formulations before Infection with Human Coronavirus 229E.
3.2.1.1 High MOI

Following 2, 3 and 4 days incubation with a high MOI of Human coronavirus 229E, the positive infection control resulted in average viral titres of 7.00, 6.50 and 4.75 Log10TCID₅₀mL⁻¹, respectively. Pre-treatment of MRC-5 cells with 1. REM and 2. IVER resulted in the highest reductions in Human coronavirus 229E recovered following incubation for 2, 3 and 4 days. (Table 4).

TABLE 4
Average recovery and reduction values for Human
coronavirus 229E following pre-treatment with two nasal sprays before
infection at a high multiplicity of infection and incubated for 2, 3 and 4 days.

| | Recovery | | | Reduction | | |
|---|---|---|---|---|---|---|
| | (Log10 TCID 50 mL⁻¹) | | | (Log10 TCID 50 mL⁻¹) | | |
| Test agent | Day 2 | Day 3 | Day 4 | Day 2 | Day 3 | Day 4 |
| Positive infection | 7.00 | 6.50 | 4.75 | N/A | N/A | N/A |
| 1. REM | 3.17 | ≤2.50 | 2.58 | 3.83 | ≥4.00 | 2.17 |
| 2. IVER | 3.50 | ≤2.50 | ≤2.50 | 3.50 | ≥4.00 | ≥2.25 |

N/A = not applicable,
NR = no reduction.

3.2.1.2 Low MOI

Following 2, 3 and 4 days incubation with a low MOI of Human coronavirus 229E, the positive infection control resulted in average viral titres of 7.50, 6.67 and 6.42 Log10TCID₅₀mL⁻¹, respectively. Pre-treatment of MRC-5 cells with 1. REM and 2. IVER resulted in the highest reductions in Human coronavirus 229E recovered following incubation for 2 and 4 days (Table 5).

TABLE 5
Average recovery and reduction values for Human
coronavirus 229E following pre- treatment with two nasal sprays before
infection at a low multiplicity of infection and incubated for 2, 3 and 4 days.

| | Recovery | | | Reduction | | |
|---|---|---|---|---|---|---|
| | (Log10 TCID 50 mL⁻¹) | | | (Log10 TCID 50 mL⁻¹) | | |
| Test agent | Day 2 | Day 3 | Day 4 | Day 2 | Day 3 | Day 4 |
| Positive infection | 7.50 | 6.67 | 6.42 | N/A | N/A | N/A |
| 1. REM | ≤2.50 | 4.50 | ≤2.50 | ≥5.00 | 2.17 | 3.92 |
| 2. IVER | 2.67 | 2.50 | 2.58 | 4.83 | ≥4.17 | 3.83 |

N/A = not applicable.

3.2.2 Treatment of Human coronavirus 229E Infected MRC-5 Cells with Two Nasal Spray Formulations
3.2.2.1 High MOI

Following 2, 3 and 4 days incubation with a high MOI of Human coronavirus 229E, the positive infection control resulted in average viral titres of 7.00, 6.50 and 4.75 Log10TCID₅₀mL⁻¹, respectively. Treatment of Human coronavirus 229E infected MRC-5 cells with 1. REM and 2. IVER resulted in the highest reductions in Human coronavirus 229E recovered following incubation for 2, 3 and 4 days (Table 6).

TABLE 6
Average recovery and reduction values for Human
coronavirus 229E following treatment with two nasal sprays after
infection at a high multiplicity of infection and incubated for 2, 3 and 4 days.

| | Recovery | | | Reduction | | |
|---|---|---|---|---|---|---|
| | (Log10 TCID 50 mL⁻¹) | | | (Log10 TCID 50 mL⁻¹) | | |
| Test agent | Day 2 | Day 3 | Day 4 | Day 2 | Day 3 | Day 4 |
| Positive infection | 7.00 | 6.50 | 4.75 | N/A | N/A | N/A |
| 1. REM | ≤2.50 | ≤2.50 | ≤2.50 | ≥4.50 | ≥4.00 | ≥2.25 |
| 2. IVER | 3.50 | ≤2.50 | ≤2.50 | 3.50 | ≥4.00 | ≥2.25 |

N/A = not applicable.
NR = no reduction.

3.2.2.2 Low MOI

Following 2, 3 and 4 days incubation with a low MOI of Human coronavirus 229E, the positive infection control resulted in average viral titres of 7.50, 6.67 and 6.42 Log10TCID₅₀mL⁻¹, respectively. Treatment of Human coronavirus 229E infected MRC-5 cells with 1. REM and 2. IVER resulted in the highest reductions in Human coronavirus 229E recovered following incubation for 2, 3 and 4 days (Table 7).

TABLE 7
Average recovery and reduction values for Human
coronavirus 229E following treatment with three nasal sprays after
infection at a low multiplicity of infection and incubated for 2, 3 and 4 days.

| | Recovery | | | Reduction | | |
|---|---|---|---|---|---|---|
| | (Log10 TCID 50 mL⁻¹) | | | (Log10 TCID 50 mL⁻¹) | | |
| Test agent | Day 2 | Day 3 | Day 4 | Day 2 | Day 3 | Day 4 |
| Positive infection | 7.50 | 6.67 | 6.42 | N/A | N/A | N/A |
| 1. REM | ≤2.50 | ≤2.50 | ≤2.50 | ≥5.00 | ≥4.17 | ≥3.92 |
| 2. IVER | ≤2.50 | ≤2.50 | ≤2.50 | ≥5.00 | ≥4.17 | ≥3.92 |

N/A = not applicable

4.0 Discussion

The dissemination of potentially pathogenic viruses increases infection risk in both healthy and immunocompromised individuals. Coronaviruses are enveloped, single stranded RNA viruses responsible for a variety of upper-respiratory tract illnesses in humans. These illnesses range from mild conditions such as the common cold to severe acute respiratory syndrome as seen in the ongoing COVID-pandemic, interventions that take both preventative and curative approaches are essential in stopping or slowing down the spread of Coronavirus. Within this study preventative and curative applications of two formulations were assessed against high and low doses of Human coronavirus 229E. Coronavirus 229E is structurally and genetically similar to the Sars-CoV-2 virus. Across all assessments REM and IVER resulted in reductions in Human coronavirus 229E recovered following preventative and curative applications.

Future work could investigate the effect of the formulations following multiple applications. Future work could also assess the nasal spray formulations against other respiratory viruses such as Influenza type A and B, Adenovirus and Rhinovirus. Bacterial respiratory pathogens such as Pseudomonas aeruginosa could also be investigated. To further mimic the real-world use of the product 3D nasal models could be used to understand the effects on ciliary function after application of the formulations.

### Preamble to Section 4

In the art, hydroxypropylmethylcellulose (HPMC) is also known by the synonym ‘Hypromellose’. The term ‘Hypromellose’ is used in the product literature of a competitor product. In order to distinguish the results of HPMC-containing powders of the applicant from that of the competitor, ‘Hypromellose’ is used in Section 4 to distinguish it from the HPMC-containing powders of the applicant.

It is to be understood that all reference to ‘Hypromellose’ within the present specification relates solely to a low pH Hypromellose containing composition of the competitor which further contains additives that act to lower the pH thereof once placed in contact with moisture. The HPMC-containing powders of the present invention do not contain additives of the kind known to be included in the competitor product.

One of the aims under Section 4 is to compare the performance of HPMC powders of the invention to the performance of the Hypromellose-containing powder of the competitor.

The Hypromellose containing product of the competitor which is being compared with HPMC-containing powders of the applicant has the following components: Hypromellose at 89.9%, citric acid at 6%, sodium citrate at 4%, benzalkonium chloride at 0.1% and menthol at <0.1% as stated in the competitor product literature.

### Section 4

Hydroxypropylmethylcellulose Gel Application Delays Der p 1 Diffusion In Vitro Significantly Better than Low pH Hypromellose

### Background:

Following updated ARIA Guidelines and data to show that certain cellulose powders can capture viral particles by forming an internal gel barrier in the nose we looked at hydroxypropylmethylcellulose powders of differing mean particle size and a commercially available low pH Hypromellose powder of a competitor for the alleviation of nasal symptoms of allergic rhinitis and for trapping viral particles including Corona virus 229E and SARS Cov2. The efficacy of these barrier compounds have been the subject of several clinical, observational, and in vitro studies. The aim of this study was to investigate the hypothesis that the quality of gel formed after moisture absorption in the nose might be related to mean particle size and that low particle size may produce a less effective barrier to external pathogens. The quality of the mechanical barrier produced by each compound will also be important in preventing allergen diffusion towards the nasal epithelium over a prolonged period of time.

Methods: The diffusion of Der p 1 through HPMC and Hypromellose gels was measured in vitro after 15, 30, 60, 180 and 360 minutes using ELISA method. Agar block were used to simulate the nasal mucosa. Control samples without gel layer were obtained.

### Section 4

Hydroxypropylmethylcellulose Gel Application Delays Der p 1 Diffusion In Vitro Significantly Better than Low pH Hypromellose

### Background:

Following updated ARIA Guidelines and data to show that certain cellulose powders can capture viral particles by forming an internal gel barrier in the nose we looked at hydroxypropylmethylcellulose powders of differing mean particle size and a commercially available low pH Hypromellose powder of a competitor for the alleviation of nasal symptoms of allergic rhinitis and for trapping viral particles including Corona virus 229E and SARS Cov2. The efficacy of these barrier compounds have been the subject of several clinical, observational, and in vitro studies. The aim of this study was to investigate the hypothesis that the quality of gel formed after moisture absorption in the nose might be related to mean particle size and that low particle size may produce a less effective barrier to external pathogens. The quality of the mechanical barrier produced by each compound will also be important in preventing allergen diffusion towards the nasal epithelium over a prolonged period of time.

Methods: The diffusion of Der p 1 through HPMC and Hypromellose gels was measured in vitro after 15, 30, 60, 180 and 360 minutes using ELISA method. Agar block were used to simulate the nasal mucosa. Control samples without gel layer were obtained.

### Results:

The control samples with no applied gel barrier absorbed 100% of the Der p 1 solution after 15 minutes. In comparison, the HPMC significantly delayed Der p 1 diffusion allowing only 1.33% penetration into agar blocks after 15 minutes and just 10.41% after 360 minutes under simulated nasal conditions with minor differences seen between small, medium and larger particles sizes and these were all superior to Hypromellose gel which allowed 5.37% penetration after 15 minutes and 25.89% after 360 minutes under same conditions.

### Conclusions:

HPMC gel significantly reduces Der p 1 diffusion in vitro compared to Hypromellose. This is likely to be due to the average mesh size of the polymer network of HPMC making a more efficient barrier than the low mesh size of Hypromellose and could have important implications for a preventative barrier formation to capture various pathogens.

### Methods

The three HPMC compounds were made up and provided for testing by Nasaleze Limited. Samples of the low pH Hypromellose compound were obtained from Nasus Pharma, IL.

Der p 1 allergen was procured from Indoor Biotechnology in India. Experimentation was followed as per a stepwise protocol as shown in FIG. 3. Followed by ELISA measurements.

### ELISA Measurements

The Der p1 allergen standards used in the assays were purchased from Indoor Biotechnologies and the assays were performed according to the manufacturer's instructions.

### Results & Observations

The mean baseline allergen content in 20 ul of the standard solution was found to be 153.02 ng following the recommended dilution and preparation of the stock solution.

Results and observations show clearly that all 3 HPMC powders are free flowing in nature when sprayed from a receptacle (conventional powder spray bottle) whereas the Hypromellose powder had to be tapped several times to get any free flow from its bottle.

All the HPMC formulations formed a thick, clear, firm gel immediately when mixed with diluents but initially the Hypromellose failed to form any kind of gel as it was in fact a liquid. Following a series of dilutions it was confirmed that all gels from the samples were made 5% gel solutions by mixing 50 mg of powder with 1 ml of 0.9% sterile saline solution to match the pH and consistency of normal nasal mucosa.

Throughout the experiment and even following 6 hours incubation at 30-35° C. the

HPMC layers remained thick in form and fresh whereas the Hypromellose layer dried up completely and formed a white precipitate on the glass slides surface.

TABLE A
Amount of Der p 1 diffused through a 1.5 mm thick HPMC or Hypromellose gel layer,
respectively, amount of allergen absorbed in ng/ml and as a percentage.

| Name of the sample & | Time in minutes | | | | |
|---|---|---|---|---|---|
| Size of the sample | 15 | 30 | 60 | 180 | 360 |
| HPMC small particulate | 2.42 ng/1.58% | 3.18 ng/2.07% | 4.26 ng/2.78% | 10.12 ng/6.61% | 18.19 ng/11.88% |
| HPMC medium particulate | 2.04 ng/1.33% | 2.62 ng/1.17% | 4.03 ng/2.63% | 8.62 ng/5.63% | 15.93 ng/10.41% |
| HPMC large particulate | 2.86 ng/1.86% | 3.63 ng/2.37% | 4.58 ng/2.99% | 10.39 ng/6.78% | 17.68 ng/11.55% |
| HYPROMELLOSE | 8.23 ng/5.37% | 11.39 ng/7.44% | 19.36 ng/12.65% | 26.34 ng/17.12% | 39.62 ng/25.89% |
| Small particulate powder | | | | | |
| Baseline Standard | 153.02 ng/100% | 153.02 ng/100% | 153.02 ng/100% | 153.02 ng/100% | 153.02 ng/100% |

pHPMC small particulate: mean particle size = 88.57 μm
pHPMC medium particulate: mean particle size = 107.7 μm
pHPMC large particulate: mean particle size = 121.00 μm
Hypromellose (low pH Hypromellose of competitor): mean particle size = 68.56 μm

### Conclusions

The data clearly show that HPMC is superior to Hypromellose of the competitor in terms of the quality, consistency and nature of the barrier produced and that this translates to HPMC being able to prevent penetration by Der p 1 allergen over a 360 minute examination being approximately 150% more effective than Hypromellose and therefore we would expect the ability to trap allergens including pollen, viruses, bacteria, and spores in the nasal mucosa to be much more efficient when using HPMC.

### REFERENCES

1. Bousquet J, Khaltaev N, Cruz A A, et al. Allergic Rhinitis and its Impact on Asthma (ARIA) 2008 update (in collaboration with the World Health Organization, GA(2)LEN and AllerGen). Allergy. April 2008; 63 Suppl 86:8-160.
2. Valerieva A, Popov T A, Staevska M, et al. Effect of micronized cellulose powder on the efficacy of topical oxymetazoline in allergic rhinitis. Allergy and asthma proceedings: the official journal of regional and state allergy societies. November-December 2015; 36(6):e134-139.
3. Diethart B, Emberlin J C, Lewis R A. Hydroxypropylmethylcellulose gel application delays Der p 1 diffusion in vitro. Natural Science. 2010;2(2):79-84.
4. Erofeeva M K, Maxakova V L, Nikonorov I Y, Pozdnyakova M G. Nasaval plus—a local mean fro protection from influenza and other acute respiratory viral infections, https://medi.ru/info/7023/. 2015.
5. Popov T A, Aberg N, Emberlin J, et al. Methyl-cellulose powder for prevention and management of nasal symptoms. Expert Rev Respir Med. November 2017; 11(11):885-892.
6. Lvov D K, Deryabin P G. Virucidal Activity of Nasaleze (Nasaval) and Nasaleze Travel (Nasaleze Plus) in Cell Cultures Infected with Pathogenic Avian Flu virus (H5N1), www.phytomedcentral.org. 2010.
7. Popov T A, Emberlin J, Josling P, Seifalian A. In vitro and in vivo Evaluation of the Efficacy and Safety of Powder Hydroxypropylmethylcellulose as Nasal Mucosal Barrier. Medical devices. 2020; 13:107-113.
8. O'Keefe B R, Giomarelli B, Barnard D L, et al. Broad-spectrum in vitro activity and in vivo efficacy of the antiviral protein griffithsin against emerging viruses of the family Coronaviridae. Journal of virology. March 2010; 84(5):2511-2521.
9. Zhou M, Zuo K J, Xu Z F, et al. Effect of Cellulose Powder on Human Nasal Epithelial Cell Activity and Ciliary Beat Frequency. International archives of allergy and immunology. Nov. 8 2018: 1-9.
10. Telko MJ, Hickey AJ. Dry powder inhaler formulation. Respiratory care. September 2005; 50(9):1209-1227.
11. Burdock GaA. Safety assessment of hydroxypropyl methylcellulose as a food ingredient. Food and chemical toxicology: an international journal published for the British Industrial Biological Research Association. December 2007; 45(12):2341-2351.
12. Andersson M, Greiff L, Ojeda P, Wollmer P. Barrier-enforcing measures as treatment principle in allergic rhinitis: a systematic review. Current medical research and opinion. June 2014; 30(6):1131-1137.
13. Josling P, Steadman S. Use of cellulose powder for the treatment of seasonal allergic rhinitis. Advances in therapy. July-August 2003; 20(4):213-219.
14. Emberlin J C, Lewis R A. A double blind, placebo-controlled cross over trial of cellulose powder by nasal provocation with Der p1 and Der f1. Current medical research and opinion. October 2007; 23(10):2423-2431.

1. A composition in the form of a dry homogenised powder consisting of two or more components selected from
i) hydroxypropyl methylcellulose particles; and
ii) at least one chemical agent selected from signalling agents; and/or
iii) one or more biologically active agents,
wherein the homogenised dry powder particles have a mean particle size of ≥20 μm to ≤500 μm.

2. A composition according to claim 1, wherein the mean particle size is in the range 60 to 150 μm.

3. A composition according to claim 1, wherein the mean particle size is in the range 80 to 125 μm.

4. A composition according to claim 2, wherein the mean particle size is 86 μm +/− 15 μm.

5. A composition according to claim 3, consisting of
i) hydroxypropyl methylcellulose particles; and
ii) at least one chemical agent selected from signalling agents.

6. A composition according to claim 3, wherein the signalling agent is selected from menthol, strawberry, mint, spearmint, peppermint, eucalyptus, lavender, citrus, and any combination thereof.

7. A composition according to claim 1, wherein the signalling agent makes up from 0.25% to ≤10% of the total weight of the composition.

8. A composition according to claim 1 consisting of
i) hydroxypropyl methylcellulose particles; and
ii) one or more biologically active agents selected from antiviral agents, antibacterial agents, and antiallergenic agents.

9. A composition according to claim 8, wherein the biologically active agent is selected from pharmaceutical, herbal, and homeopathic agents.

10. A composition according to claim 8, wherein the biologically active agent is selected from St John's Wort, valerian extract, ginkgo biloba extract, vitamins A, E or C, garlic, one or more pro-biotics, ginger, ellagic acid, echinacea, Swedish flower pollen, black walnut hulls, lemongrass, wormwood, grapefruit seed extract, broccoli, digestive enzymes, hyaluronic acid, astragalus, rosehips, gentian, hypericum, horse chestnut, ginseng, green tea, phosphatidyl serine, phosphatidyl choline, citrus, pycnogenol, caffeine, quercitin, co-enzyme Q10, yarrow, tea tree, noni juice, lipase, fructo-oligosaccharide, inulin, black cumin, stabilised allicin, or any combination thereof.

11. A composition according to claim 8, wherein the biologically active agent is an antiviral agent selected from Type I (α, β) interferons (IFN), such as IFN-β, IFNβ-1b, Type II (γ) and Type III (λ) interferons, remdesivir, ozeltamivir, zanamivir, ribavirin, lopinavir, combination of lopinavir-ritonavir and IFNβ-1b, monoclonal and (camel) polyclonal neutralising antibodies, macrolides, and plant alkaloids, or any combination thereof.

12. A composition according to claim 11, wherein the biologically active agent is selected from remdesivir and ivermectin.

13. A composition according to claim 11, wherein the antiviral agent has activity against a coronavirus species.

14. A composition according to claim 11, wherein the antiviral agent has activity against a coronavirus species selected from SARS-CoV, MERS-CoV, SARS-COV-2, HCov-NL63, HCov-OC43, CoV-HKU1, HCov-229E and mutant strains thereof.

15. A composition according to claim 8, wherein the composition provides sustained release of the biologically active agent.

16. (canceled)

17. A composition according to claim 1, wherein the said composition is for use as a nasally administered medicament.

18. A composition according to claim 1, wherein the said composition is for use in treating covid-19 disease.

19. A composition according to claim 1, wherein the said composition is for use in prophylaxis of covid-19 disease.

20. A method of making a powdered composition as defined in claim 1 for use as a medicament for treating covid-19 disease comprising:
1) adding signalling agent powder to hydroxypropyl methylcellulose powder;
2) diffusively blending the two ingredients of 1) in a blending machine; and
3) optionally adding powdered biologically active agent and further blending.

21. A method of making a powdered composition as defined in claim 1 for use as a medicament against aerial borne allergen-related disease or aerial borne pathogen disease.
